(19) United States
(12) Patent Application Publication
(10) Pub. No.: US 2015/0320582 A1
(43) Pub. Date: Nov. 12, 2015

(54) SHOULDER IMMOBILIZATION DEVICE

(71) Applicant: AKTINA CORP., Congers, NY (US)

(72) Inventors: Milan Markovic, Wayne, NJ (US); Nicholas G. Zacharopoulos, Congers, NY (US)

(73) Assignee: AKTINA CORP., Congers, NY (US)

(21) Appl. No.: 14/708,799

(22) Filed: May 11, 2015

Related U.S. Application Data
(60) Provisional application No. 61/990,791, filed on May 9, 2014.

(51) Int. Cl. A61F 5/37; A61G 1/044

(52) U.S. Cl. CPC A61F 5/3769; A61G 1/044

(57) ABSTRACT

A shoulder immobilization apparatus/device which allows simultaneous immobilization of shoulders in both anterior/posterior directions as well as superior/inferior direction using shoulder straps (belts). The straps are attached to a base board. The base board is configured to be used in conjunction with standard components including: treatment couch, head support boards, head support, thermoplastic masks, and indexing bars. The device uses only low attenuation components in the treatment area. The use of nonconductive materials makes the device suitable for use with MRI machines.

### CROSS-REFERENCE TO RELATED APPLICATIONS

The present application claims priority to U.S. Provisional Patent Application Ser. No. 61/990,791 filed May 9, 2014 and entitled “SHOULDER IMMOBILIZATION DEVICE” which is hereby incorporated herein by reference in entirety for all purposes.

### BACKGROUND

Radiation therapy treatments in the area of cervical spine and head and neck region commonly require shoulders to be pulled down (slouched) in order to expose a maximum area. There are several approaches currently used for positioning shoulders. All of the approaches require immobilization of the head. Immobilization of the head may be as simple as posterior head support and as complex as complete head immobilization.

Shoulder immobilization is commonly done in one of following three ways:
1) Shoulder restraints (see picture 2) Disadvantages of this approach: should restraints are uncomfortable (hard) and do not provide immobilization in anterior/posterior direction: Patient can easily move shoulder forward. In order to provide sufficient immobilization these parts are usually made of plastic. The material can be (and usually is) inside of the treatment field providing undesirable attenuation of the radiation during treatment block is going on here
2) Thermoplastic head and shoulder mask (see picture below). Disadvantages of this approach is complex preparation, Limited population (patients with claustrophobia cannot handle it); unknown patient position inside of the mask especially in case that patient loses weight during the course of the treatment. Major issue is emergency removal of the patient (for example if the patient starts vomiting): It requires time to release the patient.
3) Pull strap (Patients arms are attached to the foot end of the treatment couch by elastic bands providing constant pulling force). Major disadvantage is that it is not positive and requires patient's participation. This approach is very rarely used.

These existing devices on the market do not provide sufficient anterior/posterior immobilization, and have increased attenuation (components made of thicker plastic or composite materials have much higher attenuation then thin straps (similar to car seat belts). Other types of devices that provide better immobilization (such as thermoplastic sheets that cover shoulders) are patient specific: meaning more expensive and take long time to manufacture and install during treatments.

### SUMMARY

The shoulder immobilization apparatus/device disclosed allows simultaneous immobilization of shoulders in both anterior/posterior directions as well as superior/inferior direction using shoulder straps (belts). The use of separate straps for each shoulder allowing each shoulder to be independently adjusted and recorded as warranted. The straps conform to the patient's anatomy making the device more comfortable to use. Device uses only low attenuation components in the treatment area. The use of nonconductive materials makes the device suitable for use with MRI machines. The device is designed to be used in conjunction with standard components including: treatment couch, head support boards, head support, thermoplastic masks, and indexing bars.

### BRIEF DESCRIPTION OF DRAWINGS

FIG. 1 depicts the shoulder immobilization device prior to mounting to the treatment couch.

FIG. 2 depicts shoulder immobilization device mounted on the treatment couch.

FIG. 3 depicts an anterior exploded view of the shoulder immobilization device components.

FIG. 4 depicts a posterior exploded view of the shoulder immobilization device components.

FIG. 5 depicts a patient secured on the treatment couch by the shoulder immobilization device.

FIG. 6 depicts an upper view of a patient secured on the treatment couch with arrows indicating strap.

FIG. 7 depicts an exploded view of the scale on a strap.

FIG. 8 depicts a top view of the patient secured to the treatment couch.

FIG. 9 depicts another embodiment in which the straps are crossed.

FIG. 10 depicts another embodiment in which the bridge strap is connected between two shoulder straps.

FIG. 11 depicts another embodiment in which the adjustable strap is the upper portion of each shoulder strap.

FIG. 12 depicts the rear of the base board after a patient is secured to it.

### PARTS LIST

1—Base board of the shoulder immobilization device
2—Label, Indexing position
3—Adjustable shoulder strap with scale and hook and loop locking fastener
4—Pin
5—Buckle strap
6—Shoulder extension piece
7—Cover, superior
8—Cover, inferior
9—Plastic screws
10—Shoulder immobilization device
11—Head mask, thermoplastic
12—Anatomical head support
13—Head support board
14—Indexing crossbar
15—Treatment couch
16—Treatment couch indexing notch
17—Pin of the indexing crossbar
18—Indexing holes of the head support board
19—Indexing holes of the shoulder immobilization device
20—Scale of the adjustable shoulder strap
21—Angled slots for adjustable strap
22—Hook and loop cinch surfaces of the adjustable shoulder strap
23—Shoulder extension pin
24—Buckle
25—Superior Adjustable shoulder strap
26—Inferior strap with buckle
27—Bridge strap

### DETAILED DESCRIPTION

The shoulder immobilization apparatus/device [10] disclosed includes a base board [1] configured for attachment to an indexing bar [14] on a treatment couch [15] with an adjustable shoulder strap [3] on each side of a central area on the base board on which a patient's back is positioned. One end of each adjustable shoulder strap is attached to a lower portion of the base board. A shoulder extension piece [6] on each side of the central area on the base board extends away from an upper portion of the base board toward a location intended for a patient's head. A buckle strap with a buckle on one end is located on each side of the central area on the base board and configured to accept the adjustable shoulder strap. The end of each buckle strap opposite to the one end with a buckle is attached to the base board and then passed through the opening in the area of the upper end of each shoulder extension piece closest to the location intended for a patient's head and extending toward the adjustable shoulder strap.

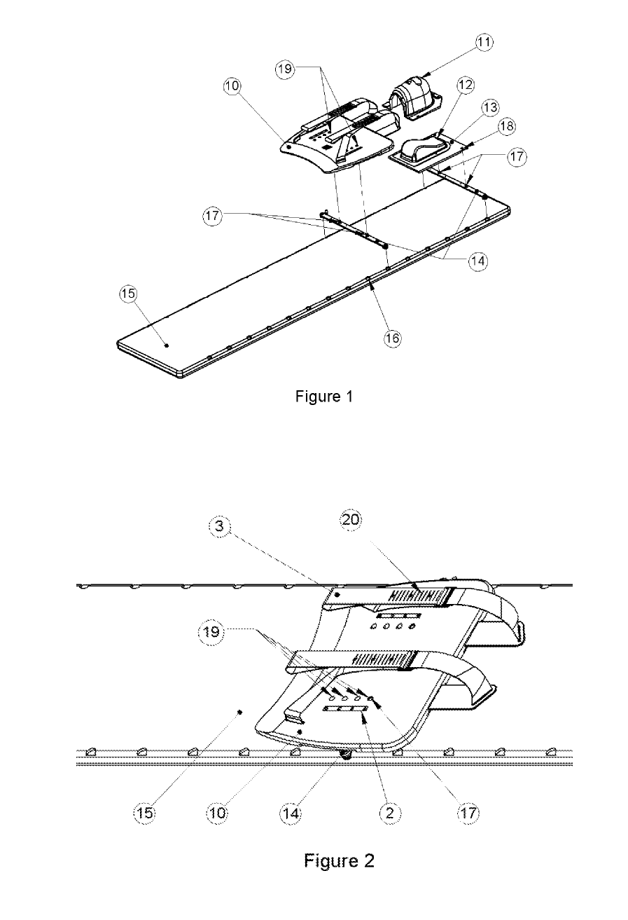

FIG. 1 shows a patient immobilization setup for Head and neck treatment. Superior and inferior indexing crossbars [14] are placed onto the treatment couch [15] in various discrete locations determined by the couch indexing notches [16]. Each notch [16] is uniquely marked in order to facilitate easy recording of its location. A head support board [13] (or similar device) is attached to the treatment couch using indexing crossbar [14] as the interface device. The head support board [13] with the anatomical head support [12] is placed over the superior indexing crossbar [14]. The pins [17] of the superior indexing crossbar [14] interlock with indexing holes [18] of the head support board. The patients head is immobilized by posterior head support [12] and anterior mask [11]. The shoulder immobilization apparatus/device [10] is attached to the treatment couch [15] using inferior indexing bar [14]. The shoulder immobilization apparatus/device [10] is placed on the inferior indexing crossbar [14] such that the pins [17] interlock with the two or more holes [19] on each side the central area on the base board [1] of the shoulder immobilization apparatus/device [10]. A indexing position label [2] is located on the base board parallel to the two or more holes on each side of the central area on the base board.

FIG. 2 shows features used to ensure a patient is accurately position during each treatment of that patient. For a particular patient the indexing bar [14] is always positioned in the same location on the treatment couch [15]. The shoulder immobilization device [10] is then positioned over the appropriate pins [17] of the indexing crossbar [14]. A label [2] is located on the shoulder immobilization device [10] to indicate into which hole [19] a pin [17] on the indexing bar [14] has been inserted to determine position of the shoulder immobilization device [10]. A patient is than placed onto the shoulder immobilization device [10] and secured using adjustable strap [3]. Adjustable strap [3] is secured in place at the position determined by the scale [20] printed on it.

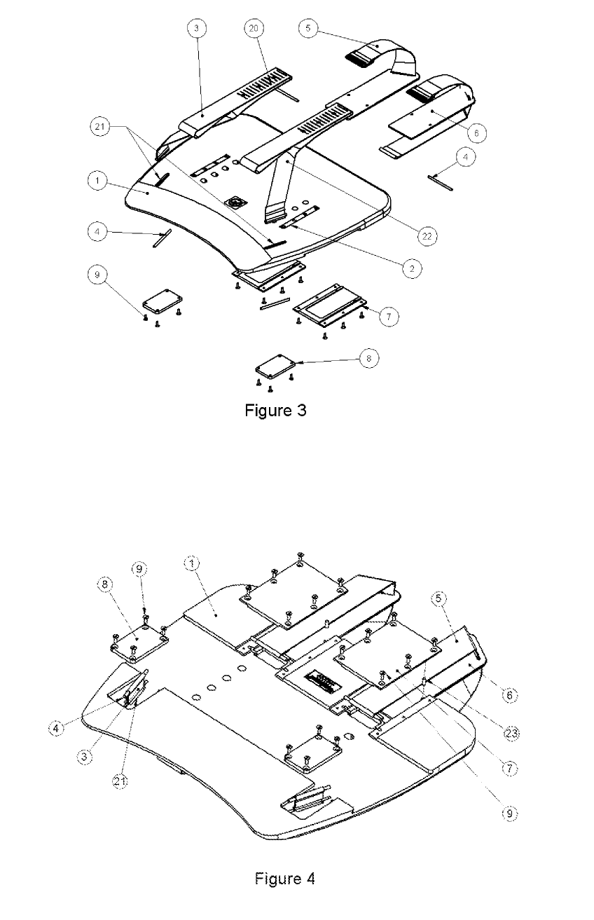

The main components of one embodiment of the shoulder immobilization device [10] are shown on FIG. 3. The base board [1] with indexing position label [2] is positioned properly on the treatment couch [15] by inserting the proper pins [17] on the indexing bar into the proper hole [19 on the base board [1]. secured to the treatment couch. Two adjustable shoulder straps [3] with scale are attached to the base board [1] after passing through an angled slot [21] in the baseboard [1]. The adjustable strap has a printed scale [20] on the portion of the strap which is visible after being secured to the buckle [24] on the buckle strap [5]. The adjustable strap may be configured to be secured to be itself after being secured to the buckle [24]. In one embodiment a hook and loop design is used to secure the belt to itself. Alternatively other methods are readily apparent to anyone skilled in the art, to secure the strap to itself such as a using a clip, magnets, or snaps.

The buckle strap [5] has a buckle on one end and the other end is attached to the base board [1] after passing through a low attenuation shoulder extension piece [6]. The shoulder extension piece is configured and positioned to extend beyond the top of the patient's shoulder. The shoulder extension piece [6] can be created as one piece with the base board or separate pieces and attached to the base board. All components of the shoulder immobilization device are made of non-conductive materials in order to permit use with MRI imaging devices.

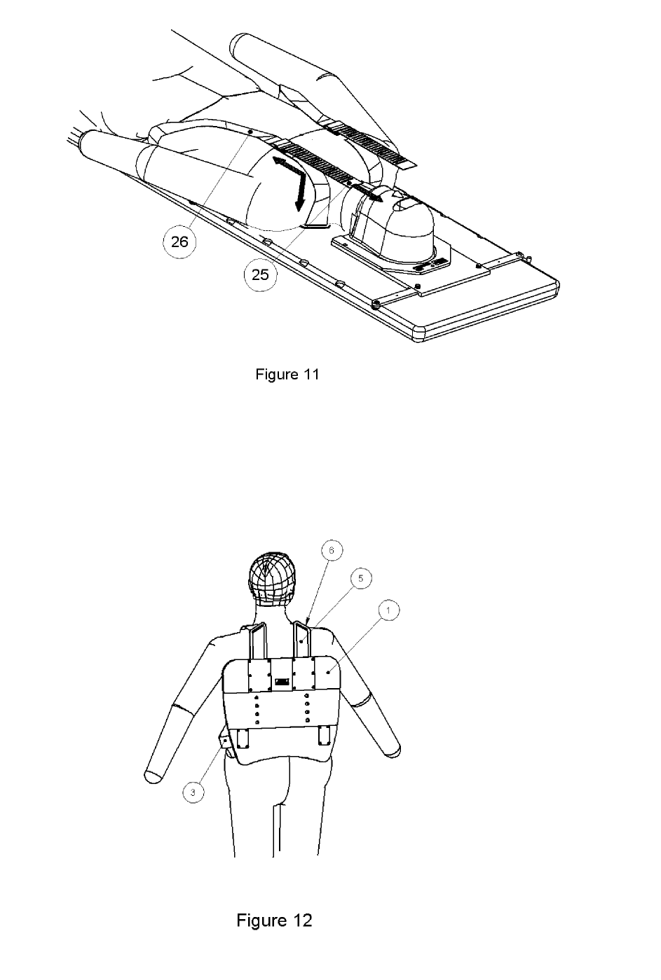

For one embodiment the components on the rear of the base board [1] are shown in an exploded view of the shoulder immobilization device on FIGS. 4 and 12. The end of the adjustable shoulder strap [3] is threaded through angled slot [21] of the base board [1]. In one embodiment the adjustable shoulder strap [3] is then secured to the bottom of the base board by inserting a pin [4] through a loop on the end of the adjustable shoulder strap [3] and inserting the pin [4] into a holder on the bottom of the base board. In another embodiment the pin [4] is than secured in place by inferior covers [8] and plastic screws [9]. The buckle strap [5] is threaded through openings of the shoulder extensions [7]. The pin [4] is placed through the end loop of the buckle strap [4]. The shoulder extension pieces can be secured to the base board using pins [23] or other well-known mechanisms to assure proper alignment of the shoulder extension pieces [6] and base board [1]. The shoulder extension piece [6], the buckle strap [5], and pins [4] and [23] may then be secured in place by a cover [7] using screws [9] or other attachments mechanisms. Numerous other configurations and devices to secure the adjustable belt and buckle belt to the base board are readily apparent to anyone skilled in the art including the use of hoop and loop strips located appropriately, clips or clamps, and a bar on the board over which a loop in the belts is positioned to secure it to the base board [1].

A patient secured to the treatment couch by the shoulder immobilization device is shown in FIG. 5. The patient's head is immobilized on the treatment couch [15] using head support [13] and thermoplastic mask 11. And the patient's shoulders are secured to the treatment couch [15] by the shoulder immobilization device [10] using the adjustable straps [3] and buckle straps [5].

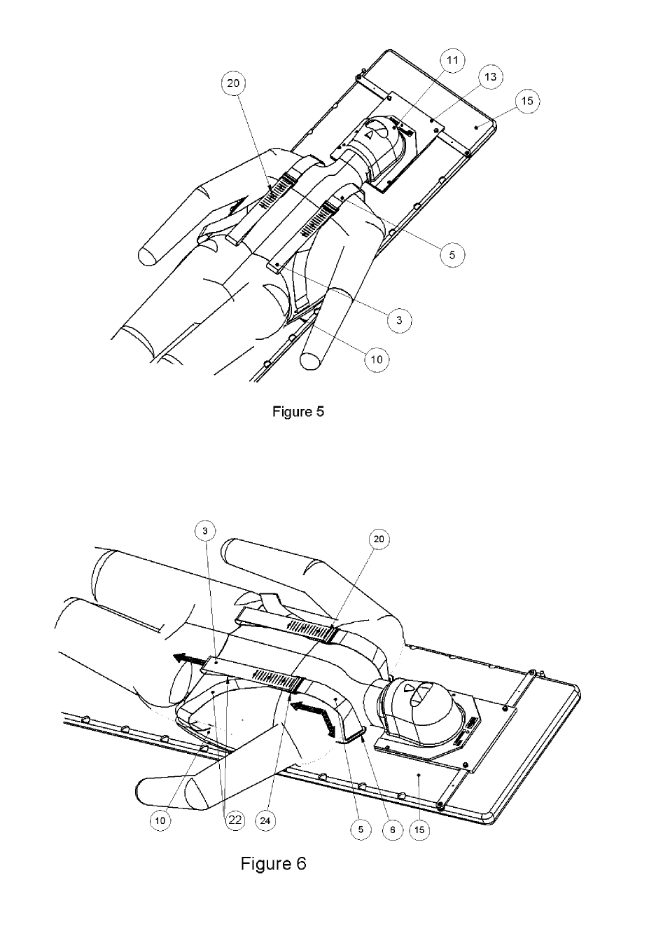

FIG. 6 shows the securing of the shoulders using shoulder immobilization device [10]. When the patient is positioned onto the treatment couch [15] with the shoulder immobilization device [10], the adjustable strap [3] is threaded through the buckle [24] of the buckle strap [5]. Pulling the adjustable strap [3] in an inferior direction away from the patient's head causes the buckle strap [3] to tighten forcing the patient shoulder in a posterior direction toward the treatment couch [15]. The shoulder extension pieces [6] are rigid ensuring the patient's shoulder is forced to move in a posterior direction towards treatment couch. The patient's head is immobilized using the mask [11], and the above described use of the shoulder immobilization device [10] causes the shoulder retraction simultaneously in both posterior and inferior direction. This causes the neck of the patient to be extended and fully exposed. Once desired position of the patient's shoulders is achieved, the adjustable strap [3] can be secured to itself as described above using hook and loop fastener surfaces [22] to interlock or other readily apparent devices. The position of the shoulders is determined by location of the scale [20]. The only components of the shoulder immobilization device [10] in the treatment area are the adjustable shoulder strap [3] and shoulder extension pieces [6]

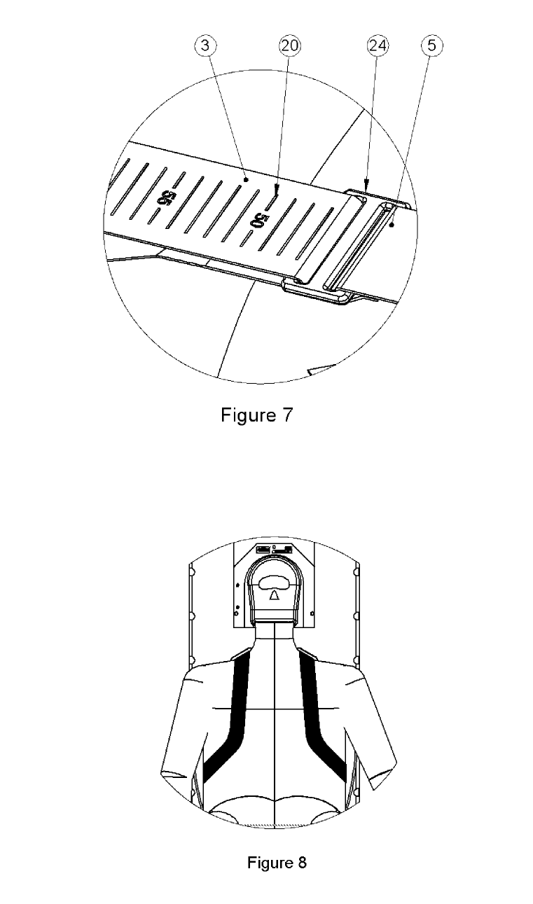

FIG. 7 is an exploded view of the scale [20] of the adjustable strap [3] after passing through the buckle [24] of the buckle strap [4]. The scale is used to record the amount of shoulder retraction during patient positioning.

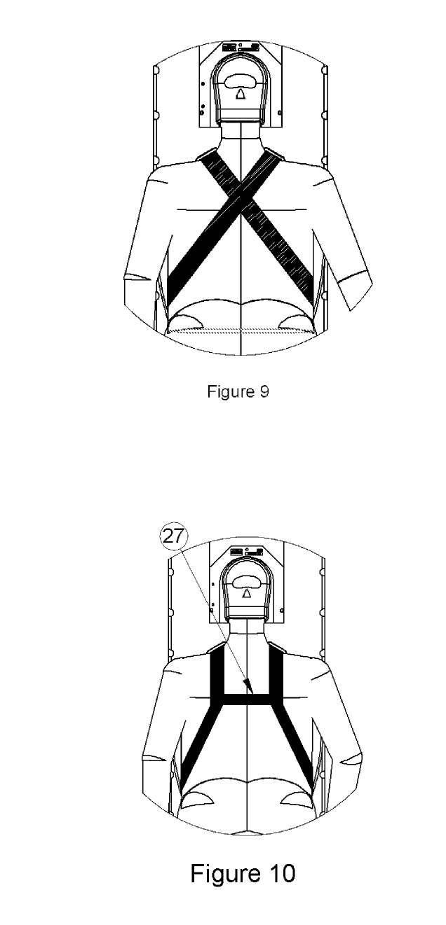

FIGS. 8, 9 and 10 show some of the possible alternate embodiments for the configuration of the shoulder straps. FIG. 8 shows strap paths parallel to the sagittal plane. FIG. 9 shows a configuration in which the straps are crossed. FIG. 10 shows strap path parallel to the sagittal plane with an added bridge strap [27] between the two adjustable straps in order to prevent sliding of the shoulder straps for patients with less pronounced shoulders. The bride strap can be configured to be adjustable to accommodate the different sizes of patients.

FIG. 11 shows another embodiment of adjustable strap location. The shoulder immobilization device can be configured with the adjustable strap [25] attached to the superior/upper portion of the device. And the buckle strap [26] configured to attach to the inferior/bottom portion of the shoulder immobilization device. This way, pulling the strap [25] in superior direction toward the head of the patient forces the shoulders to retract in inferior and posterior direction.

The above is a detailed description of particular embodiments of the invention. It is recognized that departures from the disclosed embodiments may be made within the scope of the invention and that obvious modifications will occur to a person skilled in the art. Those of skill in the art should, in light of the present disclosure, appreciate that many changes can be made in the specific embodiments which are disclosed herein and still obtain a like or similar result without departing from the spirit and scope of the invention. All of the embodiments disclosed and claimed herein can be made and executed without undue experimentation in light of the present disclosure.

We claim:

1. A shoulder immobilization device comprising:
a base board configured for attachment to a treatment couch;
an adjustable shoulder strap on each side of a central area on the base board which is intended for patient's back;
one end of each adjustable shoulder strap attached to a lower portion of the base board;
a shoulder extension piece on each side of the central area on the base board and extending away from an upper portion of the base board toward a location intended for a patient's head;
an opening in an area at an upper end of each shoulder extension piece closest to the location intended for a patient's head;
a buckle strap including a buckle on one end located on each side of the central area on the base board and configured to accept the adjustable shoulder strap;
an end of each buckle strap opposite to the one end with a buckle is attached to the base board; and
an opposite end of the buckle strap passes through the opening in the area of the upper end of each shoulder extension piece, and extends toward the adjustable shoulder strap.

2. The shoulder immobilization device according to claim 1 further comprising a scale on the adjustable strap.

3. The shoulder immobilization device according to claim 2 further comprising a mechanism to attach the adjustable strap to itself after it is captured by the buckle.

4. The shoulder immobilization device according to claim 3 in which the mechanism to attach the adjustable strap to itself is a hook and loop configuration.

5. The shoulder immobilization device according to claim 1 in which each adjustable shoulder strap passes through a slot in the base board and attached to a bottom of the base board.

6. The shoulder immobilization device according to claim 5 in which each adjustable shoulder strap is secured to the bottom of the base board by inserting a pin through a loop on an end of the adjustable shoulder strap and inserting the pin into a holder on the bottom of the base board.

7. The shoulder immobilization device according to claim 1 further comprising two or more holes in the base board on each side the central area on the base board and each hole configured to allow the insertion of a pin located on an indexing bar on the treatment couch.

8. The shoulder immobilization device according to claim 7 further comprising a scale on the base board parallel to the two or more holes on each side of the central area on the base board.

9. The shoulder immobilization device according to claim 1 further comprising a bride strap connecting the adjustable shoulder strap on each side of the base board to each other.

10. The shoulder immobilization device according to claim 1 in which the adjustable shoulder strap on each side of the central area cross each before connecting to the buckle strap.

11. The shoulder immobilization device according to claim 1 further comprising a bridge strap connecting the adjustable shoulder strap on each side of the base board to each other.

12. A shoulder immobilization device comprising:
a base board configured for attachment to an indexing bar on a treatment couch;
an adjustable shoulder strap on each side of a central area on the base board on which a patient's back is positioned;
a buckle strap with a buckle on one end located on each side of the central area on the base board and configured to accept the adjustable shoulder strap;
an end of each buckle strap opposite to the one end with a buckle is attached to the base board;
a shoulder extension piece on each side of the central area on the base board and extending away from an upper portion of the base board toward a location intended for a patient's head with an opening in an area at an upper end of each shoulder extension piece closest to the location intended for a patient's head;
an end of each adjustable shoulder strap is attached to the base board; and
an opposite end of the adjustable shoulder strap passes through the opening in the area of the upper end of each shoulder extension piece and extends toward the buckle strap.

13. The shoulder immobilization device according to claim 12 in which each buckle strap passes through a slot in the base board and attached to a bottom of the base board.

14. The shoulder immobilization device according to claim 12 further comprising two or more holes in the base board on each side the central area on the base board and each hole configured to allow the insertion of a pin located on the indexing bar.

15. The shoulder immobilization device according to claim 14 further comprising a scale on the base board parallel to the two or more holes on each side of the central area on the base board.

16. The shoulder immobilization device according to claim 12 further comprising a bridge strap connecting the adjustable shoulder strap on each side of the base board to each other.
